(19) United States
(12) Patent Application Publication
(10) Pub. No.: US 2024/0003800 A1
(43) Pub. Date: Jan. 4, 2024

(54) System for Deep Sediment Flow Culture Simulating In-situ Water Pressure

(71) Applicant: Nanjing Hydraulic Research Institute, Nanjing (CN)

(72) Inventors: Qiuwen CHEN, Nanjing (CN); Dongsheng LIU, Nanjing (CN); Jianyun ZHANG, Nanjing (CN); Haoyu ZHU, Nanjing (CN); Yuchen CHEN, Nanjing (CN); Honghai MA, Nanjing (CN); Qi ZHANG, Nanjing (CN); Lanlin WEI, Nanjing (CN)

(73) Assignee: Nanjing Hydraulic Research Institute, Nanjing (CN)

(21) Appl. No.: 18/232,405

(22) Filed: Aug. 10, 2023

Related U.S. Application Data
(63) Continuation-in-part of application No. 17/123,750, filed on Dec. 16, 2020, now abandoned.

(30) Foreign Application Priority Data
Aug. 21, 2020 (CN) 202010848160.7

(51) Int. Cl. G01N 15/08; G01N 33/18

(52) U.S. Cl. CPC G01N 15/0826; G01N 33/188

(57) ABSTRACT

A system for deep sediment flow culture simulating in-situ water pressure includes a flow culture apparatus, an inflow pressurizer and an outflow depressurizer, wherein the inflow pressurizer comprises a pressure tank, an air inlet pipe, a pressure regulating valve, a pressure-resistant container and a first support, and the pressure-resistant container containing in-situ overlying water added with isotopes is placed on the first support, a water outlet of the pressure-resistant container being connected with a water inlet pipe of the flow culture apparatus; the outflow depressurizer comprises a porous medium pipe, a second support, a depressurized water outlet pipe and a water catcher, and an outlet of the porous medium pipe is connected with one end of the depressurized water outlet pipe, the other end of the depressurized water outlet pipe extending into the water catcher.

### CROSS-REFERENCE TO RELATED APPLICATIONS

This application claims priority to U.S. application Ser. No. 17/123,750 filed 16 Dec. 2020 Chinese Patent Application Ser. No. CN202010848160.7 filed on 21 Aug. 2020.

### TECHNICAL FIELD

The present invention relates to a laboratory culture apparatus, and in particular to a system for deep sediment flow culture simulating in-situ water pressure.

### BACKGROUND

Nitrogen conversion flow culture of sediment is a water culture experiment in which in-situ overlying water added with isotopes is in a flowing state, and the nutrients, acidity and temperature of submerged sediment are kept at a stable level, which is of great significance for the simulation of nitrogen cycle processes in ecologically critical areas such as hyporheic zones of rivers and lakes, coastal dry and wet areas and hydro-fluctuation belt in reservoir areas. However, there is a big problem with existing flow culture technology, that is, the in-situ water pressure of deep sediment cannot be simulated, while water pressure is an important factor affecting nitrogen source input of sediment and release of reactant gases (Na and N₂O), which has great influence on the quantification of nitrogen cycle rate. Therefore, existing flow culture technology can hardly be applied to the culture of sediment in high-dam deep reservoirs.

### SUMMARY

Purpose: To address problems in the prior art, the present invention provides a system for deep sediment flow culture simulating in-situ water pressure.

Technical solution: The system for deep sediment flow culture simulating in-situ water pressure described herein comprises a flow culture apparatus and an inflow pressurizer and an outflow depressurizer each connected with the flow culture apparatus, wherein the inflow pressurizer comprises a pressure tank, an air inlet pipe, a pressure regulating valve, a pressure-resistant container and a first support, wherein the pressure tank is connected with an air inlet of the pressure-resistant container through the air inlet pipe, the pressure regulating valve is arranged on the air inlet pipe, and the pressure-resistant container containing in-situ overlying water added with isotopes is placed on the first support, a water outlet of the pressure-resistant container being connected with a water inlet pipe of the flow culture apparatus; the outflow depressurizer comprises a porous medium pipe, a second support, a depressurized water outlet pipe and a water catcher, wherein the porous medium pipe is filled with a porous medium material and placed on the second support, wherein an inlet of the porous medium pipe is connected with a water outlet pipe of the flow culture apparatus, and an outlet of the porous medium pipe is connected with one end of the depressurized water outlet pipe, the other end of the depressurized water outlet pipe extending into the water catcher.

Further, a pressure gauge and a pressure relief valve are arranged on the pressure-resistant container. The length, width and height of the pressure-resistant container are set as follows: length>twice the width, width>twice the height. The pressure in the pressure-resistant container is adjusted by the pressure regulating value to P during culture:

P=P₀(1+h/10)

where h is a simulated water depth and P₀ is 1 standard atmospheric pressure.

Further, the porous medium material in the porous medium pipe is a material meeting the following condition:

permeability coefficient of the porous medium material

$$K = \frac{qL}{π {r}^{2} h}$$

where q is a required flow rate of flow culture experiment, L is a length of the porous medium pipe, r is a radius of the porous medium pipe, and h is a simulated water depth.

Further, the inflow pressurizer, the flow culture apparatus and the outflow depressurizer are hermetically connected.

Beneficial effects: The present invention has significant advantages over the prior art in that the in-situ water pressure can be simulated for flow culture of deep sediment, and the outflow rate can be controlled through different media in the porous medium pipe.

### BRIEF DESCRIPTION OF THE DRAWINGS

FIG. 1 is a block diagram of a system according to one embodiment of the present invention.

### DETAILED DESCRIPTION

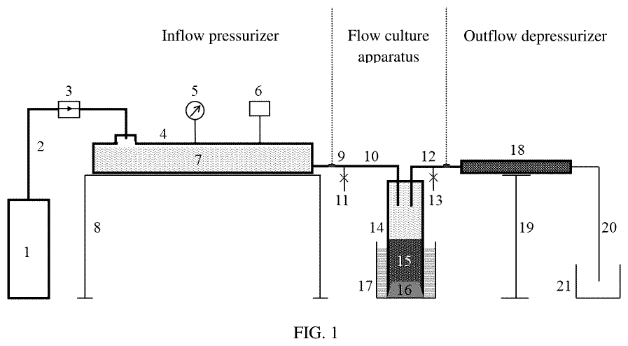

The embodiment provides a system for deep sediment flow culture simulating in-situ water pressure. As shown in FIG. 1, the system comprises a flow culture apparatus, an inflow pressurizer and an outflow depressurizer which are hermetically connected. These apparatuses are described separately below.

### (1) Inflow Pressurizer

The inflow pressurizer is configured to simulate in-situ water pressure, comprising a pressure tank 1, an air inlet pipe 2, a pressure regulating valve 3, a pressure-resistant container 4, a pressure gauge 5, a pressure relief valve 6 and a first support 8, wherein the pressure tank 1 is connected with an air inlet of the pressure-resistant container 4 through the air inlet pipe 2, the pressure regulating valve 3 is arranged on the air inlet pipe 2, and the pressure-resistant container 4 containing in-situ overlying water 7 added with isotopes is placed on the first support 8, wherein a water outlet of the pressure-resistant container 4 is hermetically connected with a water inlet pipe 10 of the flow culture apparatus. The pressure tank 1 is filled with an inert gas, and the pressure-resistant container 4 is made of steel and is integrally sealed with the water outlet. Generally, about 20-30 L of water is required for a single flow culture. In order to reduce the impact of changes of water depth in the container on simulated intensity of pressure, the container is designed with a wide and flat structure, that is, the length, width and height are set as follows: length>twice the width, width>twice the height. In the embodiment, the length, width and height are 100 cm, 30 cm and 10 cm, respectively.

During culture, it is necessary to regulate the gas injected into the pressure-resistant container 4 from the pressure tank 1 by adjusting the pressure regulating valve 3, and control the pressure of the pressure-resistant container 4 at P:

P=P₀(1+h/10)

where h is a simulated water depth and P₀ is 1 standard atmospheric pressure.

### (2) Flow Culture Apparatus

The flow culture apparatus is mainly configured for flow culture, comprising a water inlet pipe 10, an inflow sampling valve 11, a water outlet pipe 12, an outflow sampling valve 13, a flow culture pipe 14, an in-situ sediment column 15, a sealing rubber plug 16 and a thermostatic water bath pipe 17, wherein the water inlet pipe 10 is hermetically connected with the water outlet of the pressure-resistant container 4 through a hermetical connecting device 9, the inflow sampling valve 11 is located on the water inlet pipe 10, and the outflow sampling valve 13 is located on the water outlet pipe 12, both the water inlet pipe and the water outlet pipe 12 are inserted into the flow culture pipe 14, the in-situ sediment column 15 with the bottom sealed by the sealing rubber plug 16 is located in the flow culture pipe 14, and the flow culture pipe 14 is placed in the thermostatic water bath pipe 17. The water inlet pipe 10, the water outlet pipe 12 and the flow culture pipe 14 are in integrated design and made of steel, the height and inner diameter of the flow culture pipe 14 are 30 cm and 9 cm respectively, and the inner diameter of both the water inlet pipe and the water outlet pipe is 5 mm. The principle of flow culture is the same as that in the prior art, and will not be repeated here.

### (3) Outflow Depressurizer

The outflow depressurizer comprises a porous medium pipe 18, a second support 19, a depressurized water outlet pipe 20 and a water catcher 21, wherein the porous medium pipe 18 is placed on the second support 19, with its inlet being connected with the water outlet pipe 12 of the flow culture apparatus and kept at the same level, and its outlet being connected with one end of the depressurized water outlet pipe 20, the other end of which extends into the water catcher 21. The porous medium pipe 18 is made of steel and filled with a special porous medium material, which enables outflow depressurization and a low adsorption effect on the solute passing aqueous solutions. The special porous medium material is a new type of low permeability, uncharged porous polymer material, which is made by mixing metal salts and organic substance, adding anhydrous ether, and then heating for a certain period of time through a high-pressure kettle. The metal salt is Zr salt, and the organic substance is terephthalic acid. The low permeability-uncharged porous polymer material is permeable and does not have adsorption properties for ions (such as ammonium, nitrate) in the liquid.

1) Outflow depressurization: The flow rate (q) of the flow culture experiment is generally controlled at 1 ml/min. Due to the large water head difference (i.e., −h) between inflow and outflow, the present invention uses a porous medium for depressurization. According to Darcy-Weisbach Formula, the flow velocity in the porous medium pipe is:

$$v = K \frac{h}{L}$$

then permeability coefficient

$$K = \frac{qL}{π {r}^{2} h}$$

where q is a required flow rate of flow culture experiment, L is a length of the porous medium pipe, r is a radius of the porous medium pipe, and h is a simulated water depth. It can be seen that the porous medium material meets the permeability coefficient k, and the selection of the porous medium material varies with the simulated water depth (h).

To ensure the above-mentioned permeability coefficient, continuous performance testing is carried out during the production of this new porous polymer material.

2) Low adsorption effect: Experimental facilities, combined with isotope tracing and isotope pairing techniques, are used to calculate the denitrification rate and anammox rate of sediment. The water flowing into the outflow depressurizer from the flow culture apparatus is a solution of the isotopically-labeled ammonia nitrogen and nitrate nitrogen, and the concentrations of ammonia nitrogen and nitrate nitrogen are used to calculate the nitrogen conversion rate of sediment, so that the precision of the concentrations of ammonia nitrogen and nitrate nitrogen is very important for the calculation of results. Generally, the porous medium has an adsorption effect on the solute. For example, a large number of studies show that soil particles have a great adsorption effect on ammonia nitrogen. Therefore, the new porous polymer material solves this problem, as its uncharged property cannot absorb the ions (such as ammonium, nitrate) in the liquid. In addition, the system can also measure the nutrient flux at a sediment-water interface under in-situ water pressure by measuring the nutrient concentration of sediment and water before and after culture, and measure the flux of gases such as greenhouse gas released from sediment or water under in-situ water pressure by measuring the concentration of the gases such as greenhouse gas in the water before and after culture.

What is claimed is:

1. A system for deep sediment flow culture simulating in-situ water pressure, comprising a flow culture apparatus, and an inflow pressurizer and an outflow depressurizer each connected with the flow culture apparatus, wherein the inflow pressurizer comprises a pressure tank, an air inlet pipe, a pressure regulating valve, a pressure-resistant container and a first support, wherein the pressure tank is connected with an air inlet of the pressure-resistant container through the air inlet pipe, the pressure regulating valve is arranged on the air inlet pipe, and the pressure-resistant container containing in-situ overlying water added with isotopes is placed on the first support, a water outlet of the pressure-resistant container being connected with a water inlet pipe of the flow culture apparatus; the outflow depressurizer comprises a porous medium pipe, a second support, a depressurized water outlet pipe and a water catcher, wherein the porous medium pipe is filled with a porous medium material and placed on the second support, wherein an inlet of the porous medium pipe is connected with a water outlet pipe of the flow culture apparatus, and an outlet of the porous medium pipe is connected with one end of the depressurized water outlet pipe, the other end of the depressurized water outlet pipe extending into the water catcher.

2. The system for deep sediment flow culture simulating in-situ water pressure according to claim 1, wherein a pressure gauge and a pressure relief valve are arranged on the pressure-resistant container.

3. The system for deep sediment flow culture simulating in-situ water pressure according to claim 1, wherein the length, width and height of the pressure-resistant container are set as follows: length>twice the width, width>twice the height.

4. The system for deep sediment flow culture simulating in-situ water pressure according to claim 1, wherein pressure in the pressure-resistant container is adjusted by the pressure regulating value to P during culture:
P=P₀(1+h/10)
wherein h is a simulated water depth and P₀ is 1 standard atmospheric pressure.

5. The system for deep sediment flow culture simulating in-situ water pressure according to claim 1, wherein the porous medium material in the porous medium pipe is a material meeting the following condition:
permeability coefficient of the porous medium material
$$K = \frac{qL}{π {r}^{2} h}$$
wherein q is a required flow rate of flow culture experiment, L is a length of the porous medium pipe, r is a radius of the porous medium pipe, and h is a simulated water depth;
the porous medium material is low permeability, uncharged porous polymer material, which is made by mixing metal salts and organic substance, adding anhydrous ether, and then heating for a certain period of time through a high-pressure kettle, the metal salt is Zr salt, and the organic substance is terephthalic acid;
continuous performance testing for coefficient of the permeability is carried out during the production of the porous polymer material;
the porous polymer material is uncharged for reducing an adsorption effect of the porous medium material on a solute passing aqueous solutions, which achieves precision measurement of concentrations of cultured ammonia nitrogen and nitrate nitrogen, thereby reducing a systematic error in a calculation of a nitrogen conversion rate of sediment.

6. The system for deep sediment flow culture simulating in-situ water pressure according to claim 1, wherein the inflow pressurizer, the flow culture apparatus and the outflow depressurizer are hermetically connected.
